(19) United States
(12) Patent Application Publication
(10) Pub. No.: US 2007/0264304 A1
(43) Pub. Date: Nov. 15, 2007

(54) PROVISION OF VASCULAR GRAFTS WITH AN ACTIVE PRINCIPLE

(75) Inventors: KLAUS-DIETER KUHN, MARBURG-ELNHAUSEN (DE); SEBASTIAN VOGT, ERFURT (DE); MATTHIAS SCHNABELRAUCH, JENA (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH, HANAU (DE)

(21) Appl. No.: 11/696,495

(22) Filed: Apr. 4, 2007

(30) Foreign Application Priority Data
Apr. 6, 2006 (DE) 10 2006 016 598.5

(51) Int. Cl. A61K 31/7036

(52) U.S. Cl. 424/423; 514/40; 623/1.43

(57) ABSTRACT

One or more substances of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, vancomycin laurate, and vancomycin myristate are suitable for providing vascular grafts with an antithrombogenically active principle. The appropriate method for providing vascular grafts with an antithrombogenically active principle particularly comprises the following steps of A immersing the graft body in an alcoholic solution or an alcoholic solution with a readily volatile solvent, such as chloroform, wherein the alcoholic solution is of a member of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, and vancomycin myristate, or of spraying said solution on said graft body, and of B vaporizing said alcoholic solvent.

The present invention relates vascular grafts with an active principle.

In vascular surgery, large-scale use is presently made of vascular prostheses to treat vascular defects. Therein, use is particularly made of porous PTFE prostheses and knitted polyester prostheses (DACRON). After the vascular prostheses have been implanted, thrombuses may form in the area of the vascular prosthesis in the first hours after blood flow has restarted. This may impair or interrupt the blood flow, and the thrombus thus formed may be populated with bacteria. In the age of modern antibiotics, the problem of infected vascular prostheses is still a feared side-effect and presents a potentially fatal risk to the patient. It may lead to a loss of the vessel-bearing organ/sepsis and, as a consequence thereof, may cause septic shock which might result in the patient's death.

For that reason, it is desired that vascular grafts be provided with an antithrombogenic coating, so that thrombuses are effectively prevented from forming in the graft area, particularly in the first hours after implantation and before the surface of the graft starts to endothelize.

Antithrombogenic coatings, which are based on heparin, heparin derivatives and sulfated polysaccharides as well as on sulfated polysaccharide derivatives, have been disclosed in a multitude of patent applications, e.g. in CA 2510220 A1, US 2006014720 A1 or WO2005118018 A1.

The present invention aims in one embodiment at providing a coating for vascular grafts, which is able to exert an antithrombogenic effect on the porous—and also the closed—surface of vascular grafts in the presence of human blood flow for a period of several hours.

This problem is solved by using one or more substances of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, vancomycin laurate and vancomycin myristate to provide vascular grafts with an anti-thrombogenic coating.

The invention is based in part on the amazing observation that the fatty acid salts of aminoglycoside antibiotics show a distinct antithrombogenic effect, said fatty acid salts being soluble in water to a minor degree and being known as such.

According to one embodiment of the invention, porous PTFE prostheses or vascular polyester prostheses may, for example, be coated with fatty acid salts of aminoglycoside antibiotics such that the coating adheres to the PTFE while the open porous structure is preserved. It is surprising that the coated vascular prostheses maintain their necessary flexibility without any detachment of the coating.

Correspondingly, the invention also relates in another embodiment to methods for providing vascular grafts with an antithrombogenically active principle, as described hereinafter.

Optionally, further blood coagulation and/or platelet aggregation inhibitors as well as DNA or RNA or synthetic DNA analogs can be suspended in the fatty acid salts of aminoglycoside anti-biotics or incorporated in said fatty acid salts in a molecularly disperse manner without changing the coating-forming properties of said fatty acid salts, when said fatty acid salts are used according to the invention.

Such further synthetic or natural blood coagulation and/or platelet aggregation inhibitors and/or the open-chain or cyclic DNA or RNA or the synthetic DNA analogs and/or the adducts built from open-chain or cyclic DNA or RNA or synthetic DNA analogs and one or more cationic anti-biotics are enclosed in the coating either in part or as a whole. Herein, aminoglycoside antibiotics, lincosamide antibiotics and quinolone antibiotics can be used as cationic antibiotics. Therein, gentamicin, amikacin, tobramycin, clindamycin, lincosamin, ofloxacin, and moxifloxacin are particularly preferred.

There may be a further medicinal substance dispersed in the coating, wherein said medicinal substance may also be contained in the coating in a molecularly disperse manner. Argatroban, heparin and synthetically obtained polysaccharide sulfates are particularly appropriate as active antithrombogenic substance.

If used as necessary, open-chain or cyclic DNA or RNA or the synthetic analogs thereof preferrably encode growth factors or angiogenesis factors.

Therein, the fatty acid salts of aminoglycoside antibiotics simultaneously act as antithrombogenic and coating-forming substances.

### BRIEF DESCRIPTION OF THE DRAWING

The invention will now be described in greater detail with reference to the drawing, wherein:

FIG. 1 is a scanning electron micrograph of a coated vascular prosthesis according to the present invention.

The method according to the invention for providing vascular grafts with an antithrombogenically active principle particularly comprises the following steps of

immersing the graft body

A in an alcoholic solution or an alcoholic solution with a readily volatile solvent, such as chloroform,

of a member of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, and vancomycin myristate,

or of spraying said solution on said graft body, and of

B vaporizing said alcoholic solvent.

The graft body can also be sprayed with an alcoholic solution of a member of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, vancomycin laurate, and vancomycin myristate, wherein a synthetic or natural blood coagulation and/or platelet aggregation inhibitor and/or an open-chain or cyclic DNA or RNA or a synthetic DNA analog and/or one or more adducts built from open-chain or cyclic DNA or RNA or synthetic DNA analogs and one or more cationic antibiotics are suspended in said alcoholic solution, wherein the coating is formed beforehand by vaporizing the alcoholic solvent.

The thickness of the resulting coating ranges from 0.1 μm to 200 82 m.

Where PTFE or polyester is used as material for the vascular prosthesis, it is appropriate that the coating does not close the existing pore systems completely.

The invention will be illustrated by means of the following examples, without limiting the invention. Unless otherwise specified, parts or percentages refer to weight.

### EXAMPLE 1

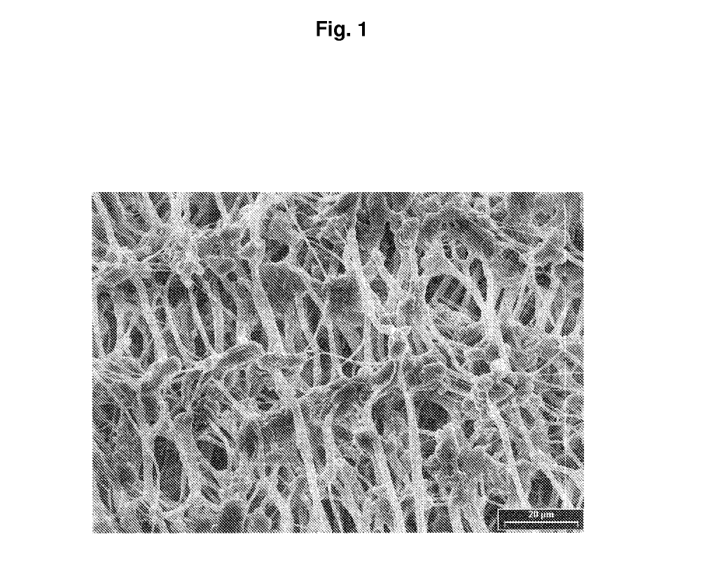

A vascular prosthesis made of expanded PTFE (6 mm in diameter) was immersed in a 5% by weight methanolic solution of gentamicin palmitate at room temperature for 60 seconds. Thereafter, the vascular PTFE prosthesis was dried at room temperature until it reached mass consistency. The applied coating of gentamicin palmitate was measured gravimetrically. Findings showed a load of 0.95 mg of gentamicin palmitate per centimeter of the vascular PTFE prosthesis. A scanning electron micrograph of the coated vascular PTFE prosthesis is shown in FIG. 1.

### EXAMPLE 2

A vascular prosthesis made of expanded PTFE (6 mm in diameter) was immersed in a 5% by weight methanolic solution of gentamicin palmitate, which contained 1% by weight argatroban, at room temperature for 60 seconds. Thereafter, the vascular PTFE prosthesis was dried at room temperature until it reached mass consistency. The applied coating of gentamicin palmitate was measured gravimetrically. Findings showed a load of 0.97 mg per centimeter.

What is claimed is:

1. A method for providing a vascular graft with an active principle, said method comprising the following steps:
A immersing a graft body in an alcoholic solution or an alcoholic solution with a volatile solvent, wherein the alcoholic solution is of a member of the group consisting of gentamicin palmitate, gentamicin myristate, gentamicin laurate, tobramycin palmitate, tobramycin myristate, tobramycin laurate, amikacin palmitate, amikacin myristate, amikacin laurate, vancomycin palmitate, and vancomycin myristate, or
spaying said graft body with said solution, and
B vaporizing said alcoholic solvent,
wherein the active principle is antithrombogenic.

2. The method according to claim 1, wherein the solution of step A has suspended therein:
a synthetic or natural blood coagulation and/or platelet aggregation inhibitor or
an open-chain or cyclic DNA or RNA or a synthetic DNA analog and/or one or more adducts built from open-chain or cyclic DNA or RNA or synthetic DNA analogs and one or more cationic antibiotics.

3. A coated vascular graft produced according to a method according to claim 1, wherein a further medicinal substance is dispersed or suspended in the coating.

4. The coated vascular graft according to claim 3, wherein the medicinal substance is contained in the coating in a molecularly dispersed manner.

5. The coated vascular graft according to claim 3, wherein the antithromogenic substance is at least one member of the group consisting of argatroban, heparin and synthetically obtained polysaccharide sulfates.

6. The coated vascular graft according to claim 3, wherein the coating contains open-chain or cyclic DNA or RNA or the synthetic analogs thereof encoding growth factors or angiogenesis factors.

7. The coated vascular graft according to claim 3, wherein the thickness of the coating ranges from 0.1 μm to 200 μm.

8. The coated vascular graft according to claim 3, wherein the graft body consists of porous PTFE or polyester.

9. The coated vascular graft according to claim 8, wherein the coating does not close any graft pore systems completely.

10. A method of treating a vascular defect in a patient in need of such treatment, said method comprising implanting a vascular graft according to claim 3 into a vein of said patient.
